(19) United States
(12) Patent Application Publication
(10) Pub. No.: US 2024/0402145 A1
(43) Pub. Date: Dec. 5, 2024

(54) SYSTEMS, METHODS, AND DEVICES FOR SOIL MOISTURE DETECTION

(71) Applicant: EarthOne Inc., Milton (CA)

(72) Inventors: Owen Patrick Purdy, Kingston (CA); Sidhanth Salvadore Pereira, Milton (CA)

(21) Appl. No.: 18/584,597

(22) Filed: Feb. 22, 2024

Related U.S. Application Data
(60) Provisional application No. 63/505,301, filed on May 31, 2023.

(51) Int. Cl. G01N 33/24

(52) U.S. Cl. CPC G01N 33/246

(57) ABSTRACT

A system, method, and device for soil moisture detection and monitoring is provided. The system comprises a plurality of soil moisture detection devices. Each soil moisture detection device comprises a processing unit; a conductive sensor configured to detect a conductive soil moisture raw data by a DC resistance measurement on a conductive voltage divider for a soil, wherein the soil operates as a variable resistor wherein a soil resistance is inversely proportional to the soil moisture; and a capacitive sensor configured to detect a capacitive soil moisture level raw data by an AC impedance measurement on a capacitive voltage divider for the soil, wherein the soil operates as a variable capacitor wherein a soil impedance is inversely proportional to a current. By providing a combination of conductive sensor and capacitive sensor, accurate soil moisture detection is provided for a variety of soil profiles.

### TECHNICAL FIELD

The following relates generally to soil moisture detection, and more particularly to systems, methods, and devices for the detection of soil moisture in multiple soil profiles.

### INTRODUCTION

Soil moisture detection and monitoring provide key advantages in the fields of agriculture, meteorology, hydrology, and environmental science.

Moisture at the root level is a critical determinant impacting plant growth, as higher or lower volumes of water may adversely affect plant health. Further, areas with limited water availability may require optimized irrigation management and diligent water use.

Soil moisture devices may provide considerable value in agriculture, supporting farmers and agriculturists in reducing water wastage. By providing accurate soil moisture measurements, the devices assist in identifying precise water requirements and reducing wastage of water resources.

In precision agriculture, farmers benefit from soil moisture detection and monitoring as part of irrigation management. By detecting irrigation levels and moisture content at the root level of the crop, moisture detection systems may provide key insights for irrigation scheduling and support irrigation management systems. As a result, the crop health is monitored and critical data is collected, which may result in maximizing the farm yield. In areas with lower water availability, soil moisture detection systems may perform as a valuable tool in efficient use of water resources. The device may provide a key advantage in supporting water management in agriculture and implementing sustainable farm practices.

Overall soil health and farm readiness may also be determined through real-time soil moisture detection. As a result, technical support may be provided for soil conservation efforts and maintaining healthy ecosystems. Real-time soil moisture detection also facilitates immediate responses to changing climatic decisions to prevent any damage to plant growth.

Soil moisture devices may also be used in other fields such as meteorology for flood and drought prediction and climate modeling. By determining soil saturation, the risk of floods may be assessed to facilitate preventive measures. Drought conditions may be monitored by assessing the dryness levels of parched soils. Climate modeling also requires soil moisture assessment to calculate the exchange of water and heat content between the soil and the atmosphere, supporting predictions in temperature change, humidity, and wind.

However, traditional soil moisture detection devices and systems do not provide accurate soil moisture detection for multiple soil types. Soils vary in moisture content and saturation for different types of soils such as coarse sand, sandy loam, loam, loamy clay, and heavy clay. Multiple soil moisture detection devices may be required for detecting moisture levels for the respective soil types.

Further, since soil moisture detection devices require electrochemical reactions, they may be susceptible to corrosion at the electrode level such as galvanic corrosion and anodic corrosion. Significant cost overheads in the maintenance of soil moisture devices may be required when installed in corrosive soils.

Accordingly, systems, methods, and devices are desired that overcome one or more disadvantages associated with existing soil moisture detection devices, methods, and systems.

### SUMMARY

A soil moisture detection system is provided comprising at least one soil moisture detection device including: a conductive sensor configured to detect a conductive soil moisture raw data by a DC resistance measurement on a conductive voltage divider for a soil, wherein the soil operates as a variable resistor wherein a soil resistance is inversely proportional to the soil moisture; a capacitive sensor configured to detect a capacitive soil moisture level raw data by an AC impedance measurement on a capacitive voltage divider for the soil, wherein the soil operates as a variable capacitor wherein a soil impedance is inversely proportional to a current; a network server configured to provide a soil moisture detection; and a user terminal configured to receive a soil moisture value.

According to an embodiment, the conductive sensor performs DC resistance measurement at a pre-set time interval.

According to an embodiment, the pre-set time interval is provided by a p-channel metal-oxide-semiconductor field-effect transistor.

According to an embodiment, the network server determines a conductive soil moisture level from:

$$soilMoistureCond = \frac{( [ V\_COND @ ( air ) ] - [ V\_COND @ ( raw ) ] )}{( [ V\_COND @ ( air ) - [ V\_COND @ ( water ) ] )}$$

wherein the soilMoistureCond refers to the conductive soil moisture level.

According to an embodiment, the network server determines a capacitive soil moisture level from:

$$soilMoistureCap = \frac{( [ V\_CAP @ ( air ) ] - [ V\_CAP @ ( raw ) ] )}{( [ V\_CAP @ ( air ) - [ V\_CAP @ ( water ) ] )}$$

wherein the soilMoistureCap refers to the capacitive soil moisture level.

According to an embodiment, the network server determines an average soil moisture level from:

$$soilMoistureAvg = \frac{( soilMoistureCap + soilMoistureCond )}{2}$$

wherein the soilMoistureAvg refers to the average soil moisture level.

According to an embodiment, the network server determines a total soil moisture level from:

$$\% soilMoistureTot = 100 * ( soilMoistureCap ( 1 - soilMoistureAvg ) + soilMoistureCond ( soilMoistureAvg )$$

wherein the % soilMoistureTot refers to the average soil moisture level.

According to an embodiment, the network server is configured to determine the conductive sensor moisture level in response to a saturated soil.

According to an embodiment, the network server is configured to determine the capacitive sensor moisture level in response to a dry soil.

A soil moisture detection device is provided comprising: a processing unit; a conductive sensor configured to detect a conductive soil moisture raw data by a DC resistance measurement on a conductive voltage divider for a soil, wherein the soil operates as a variable resistor wherein a soil resistance is inversely proportional to the soil moisture; and a capacitive sensor configured to detect a capacitive soil moisture level raw data by an AC impedance measurement on a capacitive voltage divider for the soil, wherein the soil operates as a variable capacitor wherein a soil impedance is inversely proportional to a current.

According to an embodiment, the conductive sensor performs DC resistance measurement at a pre-set time interval.

According to an embodiment, the pre-set time interval is provided by a p-channel metal-oxide-semiconductor field-effect transistor.

According to an embodiment, the processing unit determines a conductive soil moisture level from:

$$soilMoistureCond = \frac{( [ V\_COND @ ( air ) ] - [ V\_COND @ ( raw ) ] )}{( [ V\_COND @ ( air ) - [ V\_COND @ ( water ) ] )}$$

wherein the soilMoistureCond refers to the conductive soil moisture level.

According to an embodiment, the processing unit determines a capacitive soil moisture level from:

$$soilMoistureCap = \frac{( [ V\_CAP @ ( air ) ] - [ V\_CAP @ ( raw ) ] )}{( [ V\_CAP @ ( air ) - [ V\_CAP @ ( water ) ] )}$$

wherein the soilMoistureCap refers to the capacitive soil moisture level.

According to an embodiment, the processing unit determines an average soil moisture level from:

$$soilMoistureAvg = \frac{( soilMoistureCap + soilMoistureCond )}{2}$$

wherein the soilMoistureAvg refers to the average soil moisture level.

According to an embodiment, the processing unit determines a total soil moisture level from:

$$\% soilMoistureTot = 100 * ( soilMoistureCap ( 1 - soilMoistureAvg ) + soilMoistureCond ( soilMoistureAvg )$$

wherein the % soilMoistureTot refers to the average soil moisture level.

According to an embodiment, the processing unit is configured to: determine the conductive sensor moisture level in response to a saturated soil; and determine the capacitive sensor moisture level in response to a dry soil.

A soil moisture detection method is provided, comprising: collecting conductive raw voltage data at a conductive sensor by a DC resistance measurement on a conductive voltage divider for a soil, wherein the soil operates as a variable resistor wherein a soil resistance is inversely proportional to the soil moisture; collecting capacitive raw voltage data at a capacitive sensor by an AC impedance measurement on a capacitive voltage divider for the soil, wherein the soil operates as a variable capacitor wherein a soil impedance is inversely proportional to a current; and transmitting the conductive raw voltage data and capacitive raw voltage data to a server for determination of a conductive soil moisture level, a capacitive soil moisture level, an average soil moisture level, and a total soil moisture level.

According to an embodiment, the conductive soil moisture level is determined from:

$$soilMoistureCond = \frac{( [ V\_COND @ ( air ) ] - [ V\_COND @ ( raw ) ] )}{( [ V\_COND @ ( air ) - [ V\_COND @ ( water ) ] )} ;$$

the capacitive soil moisture level is determined from:

$$soilMoistureCap = \frac{( [ V\_CAP @ ( air ) ] - [ V\_CAP @ ( raw ) ] )}{( [ V\_CAP @ ( air ) - [ V\_CAP @ ( water ) ] )} ;$$

the average soil moisture level is determined from:

$$soilMoistureAvg = \frac{( soilMoistureCap + soilMoistureCond )}{2}$$

and the total soil moisture level is determined from:

$$\% soilMoistureTot = 100 * ( soilMoistureCap ( 1 - soilMoistureAvg ) + soilMoistureCond ( soilMoistureAvg )$$

wherein the soilMoistureCond refers to the conductive soil moisture level; the soilMoistureCap refers to the capacitive soil moisture level; wherein the soilMoistureAvg refers to the average soil moisture level; and the % soilMoistureTot refers to the total soil moisture level.

According to an embodiment, the server is configured to: determine the conductive sensor moisture level in response to a saturated soil; and determine the capacitive sensor moisture level in response to a dry soil.

Other aspects and features will become apparent to those ordinarily skilled in the art, upon review of the following description of some exemplary embodiments.

### BRIEF DESCRIPTION OF THE DRAWINGS

The drawings included herewith are for illustrating various examples of systems, methods, and devices of the present specification. In the drawings:

FIG. 1 is a schematic diagram illustrating a system for soil moisture, according to an embodiment;

FIG. 2 is a simplified block diagram of components of a device for real-time soil moisture detection and monitoring, according to an embodiment;

FIG. 3 is a block diagram of a device for real-time soil moisture detection and monitoring, according to an embodiment;

FIG. 4 is a flow diagram of a method for real-time soil moisture detection and monitoring, according to an embodiment;

FIG. 5 is a circuit diagram of a device for real-time soil moisture detection and monitoring, according to an embodiment; and

FIGS. 6A and 6B are schematic diagrams of printed circuit boards (PCB) of a device for real-time soil moisture detection and monitoring, according to an embodiment.

### DETAILED DESCRIPTION

Various apparatuses or processes will be described below to provide an example of each claimed embodiment. No embodiment described below limits any claimed embodiment and any claimed embodiment may cover processes or apparatuses that differ from those described below. The claimed embodiments are not limited to apparatuses or processes having all of the features of any one apparatus or process described below or to features common to multiple or all of the apparatuses described below.

One or more systems described herein may be implemented in computer programs executing on programmable computers, each comprising at least one processor, a data storage system (including volatile and non-volatile memory and/or storage elements), at least one input device, and at least one output device. For example, and without limitation, the programmable computer may be a programmable logic unit, a mainframe computer, server, personal computer, cloud-based program or system, laptop, personal data assistant, cellular telephone, smartphone, or tablet device.

Each program is preferably implemented in a high-level procedural or object-oriented programming and/or scripting language to communicate with a computer system. However, the programs may be implemented in assembly or machine language, if desired. In any case, the language may be a compiled or interpreted language. Each such computer program is preferably stored on a storage medium or a device readable by a general-or special-purpose programmable computer for configuring and operating the computer when the storage medium or device is read by the computer to perform the procedures described herein.

A description of an embodiment with several components in communication with each other does not imply that all such components are required. On the contrary, a variety of optional components are described to illustrate the wide variety of possible embodiments of the present invention.

Further, although process steps, method steps, algorithms, or the like may be described (in the disclosure and/or in the claims) in a sequential order, such processes, methods, and algorithms may be configured to work in alternate orders. Accordingly, any sequence or order of steps that may be described does not necessarily indicate a requirement that the steps be performed in that order. The steps of processes described herein may be performed in any order that is practical. Further, some steps may be performed simultaneously.

When a single device or article is described herein, it will be readily apparent that more than one device or article (whether or not they cooperate) may be used in place of a single device or article. Similarly, where more than one device or article is described herein (whether or not they cooperate), it will be readily apparent that a single device or article may be used in place of the more than one device or article.

The following relates generally to soil moisture detection, and more particularly to systems, methods, and devices for detection of soil moisture in multiple soil profiles.

Soil moisture detection systems and devices are essential tools for modern soil and plant management practices. Real-time detection of soil moisture advantageously minimizes damage to plants and protects plant growth by optimizing water use. Real-time soil moisture detection systems are designed to identify and pinpoint the moisture content at the root level, before the changes in moisture impact plant growth. By recognizing moisture imbalances at an early stage, moisture detection systems provide invaluable data to farmers so that they take informed decisions in irrigation management. Real-time soil moisture detection also helps maximize water resource allocation in a variety of soil types. As such, real-time soil moisture detection and monitoring systems play a key role in supporting plant growth, while safeguarding environments, water resources, and energy.

The present disclosure provides an IoT (Internet of Things)-based device with at least one conductive sensor and one capacitive sensor designed to provide accurate results for heterogeneous soil profiles including dry, moderate, or wet soil conditions. By providing real-time data on soil moisture levels, the device may assist users such as agriculturists, gardeners or farmers to make more informed decisions about irrigation, water delivery, and other plant management and health monitoring practices.

The soil moisture detection device includes at least one conductive soil sensor and at least one capacitive soil sensor. By providing a combination of sensors including at least one conductive soil sensor and at least one capacitive oil sensor, enhanced moisture detection capability is achieved for soils ranging from highly saturated to dry or parched.

The conductive soil sensor is configured to measure the moisture content of the soil by performing a DC resistance measurement using a voltage divider. The soil is represented as a variable resistor which has increased resistance as moisture decreases. Performing the DC resistance measurement requires current flow across the two contacts through the soil. While the current is flowing, the oxide is deposited onto the anodic contact on the PCB (Printed Circuit Board). To minimize oxide buildup, the circuit is turned on using a pMOS transistor (p-channel metal-oxide-semiconductor field-effect transistor) for approximately 1 microsecond during each data collection cycle.

According to an embodiment, the soil moisture detection device is connected to a microcontroller. The microcontroller is configured to provide one or more of the data processing, data storage, circuit control, communication, automation, task timing and scheduling functions. For example, an ESP32 microcontroller is connected to the soil moisture detection device.

According to an embodiment, the data collection cycle in the soil moisture detection device is adjusted by the microcontroller. Preferably, the data collection cycle is set at 1 microsecond. The duration of the data collection cycle is configurable and may be adjusted through a software interface or a mobile application communicatively connected to the microcontroller. The circuit activation of 1 microsecond is controlled by the pMOS transistor further connected to the microcontroller. The pMOS transistor on receiving instructions from the microcontroller, is configured to pulled up to logic level high or pulled down to a logic level low (0V) to activate or deactivate the circuit based on the data collection cycle. For example, the 1-microsecond data collection cycle may be e-programmatically changed in the code on the microcontroller.

To minimize oxide buildup, the data collection cycle is activated once every data recording period. For example, the data collection cycle of 1 microsecond is activated once every data recording period of 10 minutes. The data recording period is adjustable through the microcontroller.

The capacitive soil sensor is configured to measure the moisture content of the soil by performing an AC impedance measurement. This is performed using the change in the circuit impedance caused by soil's variable capacitance to measure the corresponding change in voltage across the resistor. The soil is represented as a variable capacitor which has increased impedance as current decreases.

According to an embodiment, on the printed circuit board, the capacitive pads are covered by a solder mask. The conductive patches are exposed. The conductive and capacitive sensors are placed on the bottom portion of the printed circuit board. The bottom portion is inserted into the soil for moisture detection.

According to an embodiment, the capacitive sensor is covered with a corrosion resistant material to prevent corrosion.

According to an embodiment, the capacitive sensor is activated in synchronization with the data collection cycle and data recording period in the conductive sensor.

The soil moisture values collected by the conductive sensor and the capacitive sensor are collected as raw voltages. During the operation, in the conductive soil sensor, the conductance is directly proportional to the moisture content in the soil. As a result, the voltage level collected by the conductive sensor will be higher for a soil with high moisture content. During the operation, in the capacitive soil sensor, the capacitance is directly proportional to the soil moisture content. According to an embodiment, the raw voltages are converted into a percentage value through logical processing and equations on the server. For example, 100% soil moisture indicates that the sensor is in water, and 0% soil moisture indicates the sensor is in the air. Similarly, 10% soil moisture indicates a comparatively dry soil with low moisture content, and 90% soil moisture indicates a comparatively wet soil with high moisture content.

According to an embodiment, a method for real-time soil moisture detection and monitoring is provided.

Considering the improved performance of a conductive soil sensor in highly saturated soil, and the capacitive soil sensor in dry or parched soil, the method includes calculation of the percentage of soil moisture in the soil weights, the conductive sensor higher in saturated conditions, and the capacitive sensor higher in dry conditions. The calculation is mathematically represented as:

$$soilMoistureCap = \frac{( [ V\_CAP @ ( air ) ] - [ V\_CAP @ ( raw ) ] )}{( [ V\_CAP @ ( air ) - [ V\_CAP @ ( water ) ] )} \qquad ( 1 )$$

$$soilMoistureCond = \frac{( [ V\_COND @ ( air ) ] - [ V\_COND @ ( raw ) ] )}{( [ V\_COND @ ( air ) - [ V\_COND @ ( water ) ] )} \qquad ( 2 )$$

$$soilMoistureAvg = \frac{( soilMoistureCap + soilMoistureCond )}{2} \qquad ( 3 )$$

$$\% soilMoistureTot = 100 * ( soilMoistureCap ( 1 - soilMoistureAvg ) + soilMoistureCond ( soilMoistureAvg ) \qquad ( 4 )$$

“V_CAP@(air)” indicates the raw voltage calculated by the capacitive sensor in the air i.e., where no or negligible moisture is present. According to an embodiment, this value may be recorded once and used for future calculations and logic processing.

“V_CAP@(raw)” indicates the raw voltage calculated by the capacitive sensor in the soil for which the overall moisture is required to be detected.

“V_CAP@(water)” indicates the raw voltage calculated by the capacitive sensor in the water i.e., where highest moisture levels are recorded. According to an embodiment, this value may be recorded once and used for future calculations and logic processing.

“soilMoistureCap” indicates the soil moisture value for the soil.

“V_COND@(air)” indicates the raw voltage calculated by the conductive sensor in the air i.e., where no or negligible moisture is present. According to an embodiment, this value may be recorded once and used for future calculations and logic processing.

“V_COND@(raw)” indicates the raw voltage calculated by the conductive sensor in the soil for which the overall moisture is required to be detected.

“V_COND@(water)” indicates the raw voltage calculated by the conductive sensor in the water i.e., where highest moisture levels are recorded. According to an embodiment, this value may be recorded once and used for future calculations and logic processing.

“soilMoistureCond” indicates the soil moisture value for the soil.

“soilMoistureAvg” indicates the weighted average of the soil moisture based on the soil moisture calculated by the conductive sensor and the soil moisture calculated by the capacitive sensor.

“% soilMoistureTot” indicates the percentage total soil moisture level in the soil.

According to an embodiment, both the capacitive sensor and the conductive sensor are active in a soil detection operation. The weight accorded to each sensor is adjusted based on the individual sensor measurements and weighing method prestored on the server database.

In the calculation operation, a temporary average between the sensors is performed, which is then used to weigh each of the individual values. The values of both soil moisture sensors are then combined using the weighting as shown in Equation 4. In 100% saturated soil, only the conductive sensor is used. In 0% saturated soil, only the capacitive sensor is used. In 50% saturated soil, the two sensors are weighted equivalently.

According to an embodiment, the method further includes applying a plurality of weighted methods and technical mathematical expressions to calculate the soil moisture levels.

According to an embodiment, the method further includes controlling electricity pulses at pre-set periods to improve accuracy. According to an embodiment, the conductive sensor takes 5 measurements to reduce variance. The number of measurements required to reduce variance can be programmatically adjusted.

The device further includes a data collection module to collect soil moisture data, a microcontroller, a power source, and a wireless communication module.

According to an embodiment, the microcontroller is an ESP32 microcontroller.

According to an embodiment, the power source is an 3.7-4.2V lithium ion battery.

According to an embodiment, a communication system for the soil moisture detection and monitoring device is provided.

The soil moisture detection and monitoring device is assigned an identifier which is recognized by a central database in the cloud server.

The soil moisture detection and monitoring devices record both capacitance and conductive values in raw voltages. The raw voltage values are then transmitted to the cloud at set polling rates.

According to an embodiment, the raw voltage levels are processed on the server to determine the overall soil moisture levels and percentage.

According to an embodiment, the preferable range for polling rates is 10 minutes to 60 minutes.

EarthOne's servers store the raw voltage values as they are entered under the USER ID, DEVICE ID, and PLANT ID.

According to an embodiment, the USER ID indicates the account registration identifier such as username, email address, or mobile number. The account registration refers to an account on the digital platform such as a mobile application to receive processed data from the soil moisture detection device.

According to an embodiment, the PLANT ID and the DEVICE ID are provided by the administrator and the IDs are associated with the USER ID.

The combined algorithm or method is performed in the cloud using the raw voltages as inputs.

The resulting percentage values are stored under the relevant USER ID and PLANT ID of the registered device.

The resulting percentage values are transmitted to the user terminal via the dashboard under their specific plant on their EarthOne Grow mobile application.

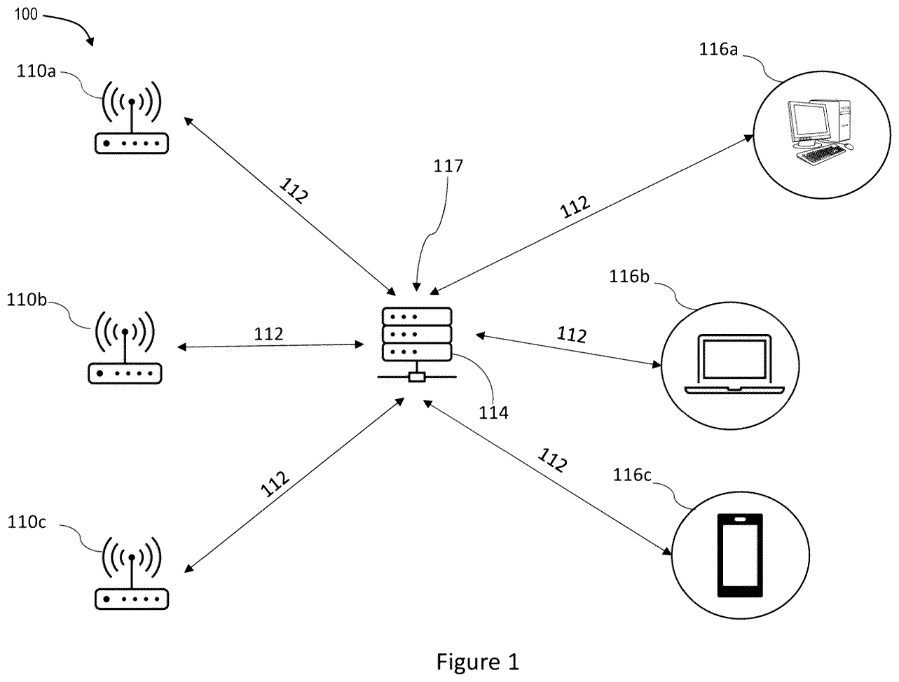

Referring now to FIG. 1, shown therein is a schematic diagram illustrating a system 100 for real-time detection and monitoring of soil moisture, according to an embodiment.

The system 100 includes an IoT-based plurality of soil moisture detection and monitoring devices 110a, 110b, and 110c (collectively referred to as soil moisture detection devices 110 and generically referred to as the soil moisture detection device 110), a network 112 to provide communication between the components of the system 100, a network server 114 to provide network services including data processing, storage, application and device management, and resource sharing, and, a plurality of terminals 116a, 116b, and 116c (collectively referred to as the terminals 116 and generically referred to as the terminal 116) for running soil moisture detection applications, and a processing station 117 (not shown) for providing data services.

According to an embodiment, the terminals 116a, 116b, and 116c, are configured as a combination of user terminals and administrator terminals.

The network 112 may be configured as a wired, wireless, or hybrid (partially wired and wireless) network based on a type of communication link used for connecting devices. The wired network 112 may include physical cables, such as Ethernet cables, to connect components in the system 100. The wireless network 112 may include Wi-Fi, Wi-Max, RFID, or Bluetooth functionality to connect components in the system 100. The hybrid network may include a combination of wired and wireless networks. Ethernet connections may be made between switches and routers (not shown) to provide wireless connections between the terminals 120 using wireless connections.

According to an embodiment, the soil moisture detection device 110 includes at least one conductive soil sensor and at least one capacitive soil sensor. By providing a combination of sensors including at least one conductive soil sensor and at least one capacitive oil sensor, enhanced moisture detection capability is achieved for soils ranging from highly saturated to dry or parched.

The conductive soil sensor is configured to measure the moisture content of the soil by performing a DC resistance measurement using a voltage divider. The soil is represented as a variable resistor which has increased resistance as moisture decreases. Performing the DC resistance measurement requires current flow across the two contacts through the soil. While the current is flowing, the oxide is deposited onto the anodic contact on the PCB (Printed Circuit Board). To minimize oxide buildup, the circuit is turned on using a pMOS transistor (p-channel metal-oxide-semiconductor field-effect transistor) for approximately 1 microsecond during each data collection cycle.

According to an embodiment, the soil moisture detection device is connected to a microcontroller. The microcontroller is configured to provide one or more of the data processing, data storage, circuit control, communication, automation, task timing and scheduling functions. For example, an ESP32 microcontroller is connected to the soil moisture detection device.

According to an embodiment, the data collection cycle in the soil moisture detection device is adjusted by the microcontroller. Preferably, the data collection cycle is set at 1 microsecond. The duration of the data collection cycle is configurable and may be adjusted through a software interface or a mobile application communicatively connected to the microcontroller. The circuit activation of 1 microsecond is controlled by the pMOS transistor further connected to the microcontroller. The pMOS transistor on receiving instructions from the microcontroller, is configured to pulled up to logic level high or pulled down to a logic level low (0V) to activate or deactivate the circuit based on the data collection cycle. For example, the 1-microsecond data collection cycle may be e-programmatically changed in the code on the microcontroller.

To minimize oxide buildup, the data collection cycle is activated once every data recording period. For example, the data collection cycle of 1 microsecond is activated once every data recording period of 10 minutes. The data recording period is adjustable through the microcontroller.

The capacitive soil sensor is configured to measure the moisture content of the soil by performing an AC impedance measurement. This is performed using the change in the circuit impedance caused by soil's variable capacitance to measure the corresponding change in voltage across the resistor. The soil is represented as a variable capacitor which has increased impedance as current decreases.

The soil moisture values collected by the conductive sensor and the capacitive sensor are collected as raw voltages. During the operation, in the conductive soil sensor, the conductance is directly proportional to the moisture content in the soil. As a result, the voltage level collected by the conductive sensor will be higher for a soil with high moisture content. During the operation, in the capacitive soil sensor, the capacitance is directly proportional to the soil moisture content. According to an embodiment, the raw voltages are converted into a percentage value through logical processing and equations on the server. For example, 100% soil moisture indicates that the sensor is in water, and 0% soil moisture indicates the sensor is in the air. Similarly, 10% soil moisture indicates a comparatively dry soil with low moisture content, and 90% soil moisture indicates a comparatively wet soil with high moisture content.

According to an embodiment, the soil moisture detection device 110 may transmit raw voltage data relating to the presence and quantity of soil moisture to server 114.

According to an embodiment, the processing station 117 may be integrated with the network server 114 as shown in FIG. 1. The processing station 117 provides data services including receiving, analyzing, and processing data received from network 112.

According to an embodiment, the network server 114 may be configured as a cloud server.

According to an embodiment, network server 114 may be configured as an application server for running soil moisture detection applications. The application server may provide services including web application hosting, resource management, connection pooling, memory allocation, load balancing, data transaction management, data access, application logic, database management, business logic processing, interoperability services, API integration, and security such as encryption and data authentication.

The terminal 116 include computer terminals for accessing the processed data from the soil moisture detection system 100, for example, outputs of the network server 114. The terminal 116 may include mobile devices, smartphones, tablets, desktop computers, laptops, thin clients, kiosks, data processing terminals, and workstations.

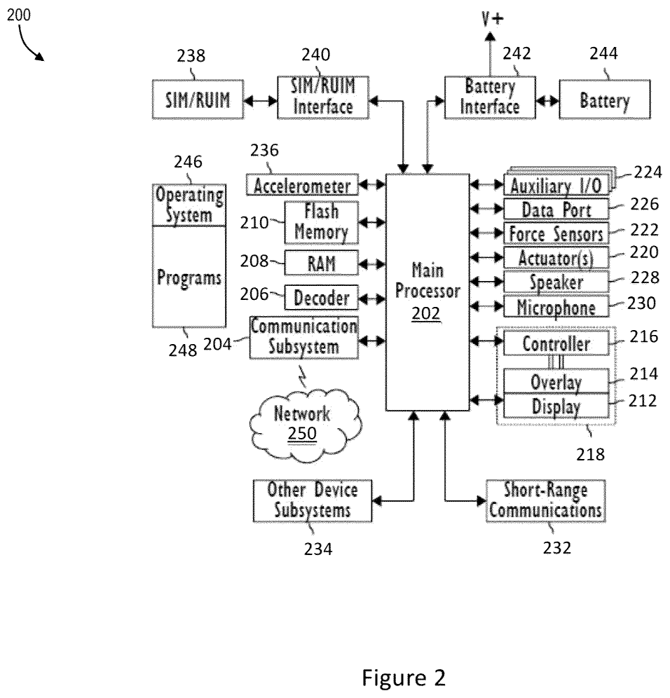

Referring now to FIG. 2, shown therein is a simplified block diagram of components of a device 200, according to an embodiment. The device 200 may correspond to any of the soil moisture detection devices 110 shown in FIG. 1. The device 200 includes a processor 202 that controls the operations of the device 200. Communication functions, including data communications, voice communications, or both may be performed through a wireless communication subsystem 204. The communication subsystem may be a wireless connection module in the soil moisture detection device 110. The communication subsystem 204 may receive messages from, and send messages to, a wireless network 250. The wireless network may be the network 112 in FIG. 1. Data received by the device 200 may be decompressed and decrypted by a decoder 206.

The wireless network 250 may be any type of wireless network, including, but not limited to, data-centric wireless networks, voice-centric wireless networks, and dual-mode networks that support both voice and data communications.

The device 200 may be a battery-powered device and as shown includes a battery interface 242 for connecting to one or more rechargeable or non-rechargeable batteries 244. The device 200 may include a power supply assembly (not shown). The device 200 may further include one or more non-rechargeable batteries (not shown).

The processor 202 also interacts with additional subsystems such as a Random Access Memory (RAM) 208, a flash memory 210, a display 212 (e.g. with a touch-sensitive overlay 214 connected to an electronic controller 216 that together comprise a touch-sensitive display 218), an actuator assembly 220, one or more optional force sensors 222, an auxiliary input/output (I/O) subsystem 224, a data port 226, a speaker 228, a microphone 230, short-range communications systems 232 and other device subsystems 234.

In some embodiments, user-interaction with the graphical user interface may be performed through the touch-sensitive overlay 214. The processor 202 may interact with the touch-sensitive overlay 214 via the electronic controller 216. Information, such as text, characters, symbols, images, icons, and other items that may be displayed or rendered on a portable electronic device generated by the processor 202 may be displayed on the touch-sensitive display 218.

The processor 202 may also interact with an accelerometer 236 as shown in FIG. 2. The accelerometer 236 may be utilized for detecting direction of gravitational forces or gravity-induced reaction forces.

To identify a subscriber for network access according to the present embodiment, the device 200 may use a Subscriber Identity Module or a Removable User Identity Module (SIM/RUIM) card 238 inserted into a SIM/RUIM interface 240 for communication with a network (such as the wireless network 250). Alternatively, user identification information may be programmed into the flash memory 210 or performed using other techniques.

The device 200 also includes an operating system 246 and software components 248 that are executed by the processor 202 and which may be stored in a persistent data storage device such as the flash memory 210. Additional applications may be loaded onto the device 200 through the wireless network 250, the auxiliary I/O subsystem 224, the data port 226, the short-range communications subsystem 232, or any other suitable device subsystem 234.

For example, in use, a received signal such as a text message, an e-mail message, web page download, or other data may be processed by the communication subsystem 204 and input to the processor 202. The processor 202 then processes the received signal for output to the display 212 or alternatively to the auxiliary I/O subsystem 224. A subscriber may also compose data items, such as e-mail messages, for example, which may be transmitted over the wireless network 250 through the communication subsystem 204.

For voice communications, the overall operation of the device 200 may be similar. The speaker 228 may output audible information converted from electrical signals, and the microphone 230 may convert audible information into electrical signals for processing.

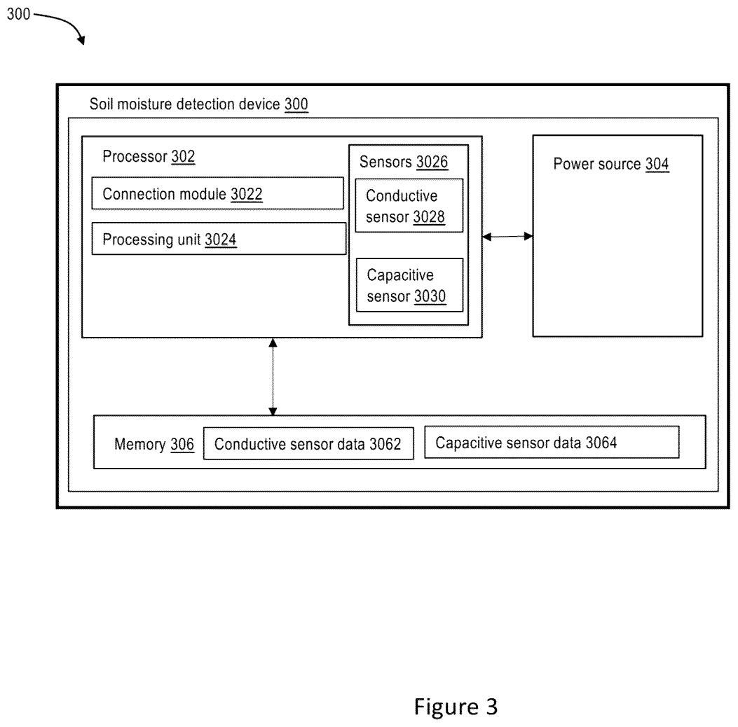

Referring now to FIG. 3, shown therein is a block diagram of a soil moisture detection device 300, according to an embodiment. The soil moisture detection device 300 may be a soil moisture detection device 110 of FIG. 1.

The soil moisture detection device 300 includes a processor 302, power source 304, and memory 306.

The processor 302 includes a connection module 3022 for providing connectivity services, a processing unit 3024 to execute instructions, and a plurality of sensors 3026. The plurality of sensors 3026 includes a conductive sensor 3028 and a capacitive sensor 3030.

The power source 304 may include a power storage to store and provide electrical power, a charging unit, and a circuit to provide control of the electrical current.

The connection module 3022 may be configured to connect the soil moisture detection device 300 to the network 112 of FIG. 1 to enable data transmission and reception therebetween. The connection module 3022 may be configured to convert received radio frequency signals and sensed values into digital data. The connection module 3022 may include an antenna configured to convert the sensed values into electromagnetic waves for transmission. The connection module 3022 may be configured to connect the components within the soil moisture detection device 300, including the processing unit 3024, sensors 3026, power source 304, and memory 306.

The processor 302 includes a plurality of sensors 3028. The plurality of sensors 3028 include at least one conductive soil sensor and at least one capacitive soil sensor. By providing a combination of sensors including at least one conductive soil sensor and at least one capacitive oil sensor, enhanced moisture detection capability is achieved for soils ranging from highly saturated to dry or parched.

The conductive soil sensor is configured to measure the moisture content of the soil by performing a DC resistance measurement using a voltage divider. The soil is represented as a variable resistor which has increased resistance as moisture decreases. Performing the DC resistance measurement requires current flow across the two contacts through the soil. While the current is flowing, the oxide is deposited onto the anodic contact on the PCB (Printed Circuit Board). To minimize oxide buildup, the circuit is turned on using a pMOS transistor (p-channel metal-oxide-semiconductor field-effect transistor) for approximately 1 microsecond during each data collection cycle.

The capacitive soil sensor is configured to measure the moisture content of the soil by performing an AC impedance measurement using a voltage divider. The soil is represented as a variable capacitor which has increased impedance as current decreases.

The sensed data may thereafter be transmitted to the processing unit 3024.

According to an embodiment, the sensed data may be stored in the memory 306 as the conductive sensor data 3062 and capacitive sensor data 3064. The sensor data relates to the presence and quantity of soil moisture in the proximity of the sensor location.

According to an embodiment, the power source 304 may include a plurality of batteries. The power source may be a non-rechargeable or rechargeable battery.

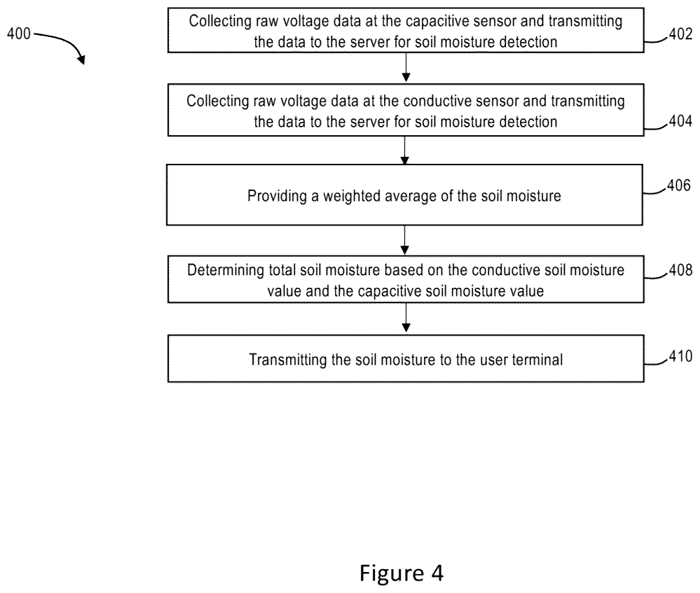

Referring now to FIG. 4, shown therein is a flow diagram of a method for real-time soil moisture detection and monitoring, according to an embodiment.

At 402, the raw voltage data at the capacitive sensor is collected and transmitted to the server for soil moisture detection.

The capacitive soil sensor is configured to measure the moisture content of the soil by performing an AC impedance measurement. This is performed using the change in the circuit impedance caused by soil's variable capacitance to measure the corresponding change in voltage across the resistor. The soil is represented as a variable capacitor which has increased impedance as current decreases.

According to an embodiment, the soil moisture is detected from the raw voltage data at the capacitive sensor by the following:

$$soilMoistureCap = \frac{( [ V\_CAP @ ( air ) ] - [ V\_CAP @ ( raw ) ] )}{( [ V\_CAP @ ( air ) - [ V\_CAP @ ( water ) ] )}$$

“V_CAP@(air)” indicates the raw voltage calculated by the capacitive sensor in the air i.e., where no or negligible moisture is present. According to an embodiment, this value may be recorded once and used for future calculations and logic processing.

“V_CAP@(raw)” indicates the raw voltage calculated by the capacitive sensor in the soil for which the overall moisture is required to be detected.

“V_CAP@(water)” indicates the raw voltage calculated by the capacitive sensor in the water i.e., where highest moisture levels are recorded. According to an embodiment, this value may be recorded once and used for future calculations and logic processing.

“soilMoistureCap” indicates the soil moisture value for the soil.

At 404, the raw voltage data at the conductive sensor is collected and transmitted to the server for soil moisture detection.

The conductive soil sensor is configured to measure the moisture content of the soil by performing a DC resistance measurement using a voltage divider. The soil is represented as a variable resistor which has increased resistance as moisture decreases. Performing the DC resistance measurement requires current flow across the two contacts through the soil. While the current is flowing, the oxide is deposited onto the anodic contact on the PCB (Printed Circuit Board). To minimize oxide buildup, the circuit is turned on using a pMOS transistor (p-channel metal-oxide-semiconductor field-effect transistor) for approximately 1 microsecond during each data collection cycle.

According to an embodiment, the soil moisture detection device is connected to a microcontroller. The microcontroller is configured to provide one or more of the data processing, data storage, circuit control, communication, automation, task timing and scheduling functions. For example, an ESP32 microcontroller is connected to the soil moisture detection device.

According to an embodiment, the data collection cycle in the soil moisture detection device is adjusted by the microcontroller. Preferably, the data collection cycle is set at 1 microsecond. The duration of the data collection cycle is configurable and may be adjusted through a software interface or a mobile application communicatively connected to the microcontroller. The circuit activation of 1 microsecond is controlled by the pMOS transistor further connected to the microcontroller. The pMOS transistor on receiving instructions from the microcontroller, is configured to pulled up to logic level high or pulled down to a logic level low (0V) to activate or deactivate the circuit based on the data collection cycle. For example, the 1-microsecond data collection cycle may be e-programmatically changed in the code on the microcontroller.

To minimize oxide buildup, the data collection cycle is activated once every data recording period. For example, the data collection cycle of 1 microsecond is activated once every data recording period of 10 minutes. The data recording period is adjustable through the microcontroller.

According to an embodiment, the soil moisture is detected from the raw voltage data at the conductive sensor by the following:

$$soilMoistureCond = \frac{( [ V\_COND @ ( air ) ] - [ V\_COND @ ( raw ) ] )}{( [ V\_COND @ ( air ) - [ V\_COND @ ( water ) ] )}$$

“V_COND@(air)” indicates the raw voltage calculated by the conductive sensor in the air i.e., where no or negligible moisture is present. According to an embodiment, this value may be recorded once and used for future calculations and logic processing.

“V_COND@(raw)” indicates the raw voltage calculated by the conductive sensor in the soil for which the overall moisture is required to be detected.

“V_COND@(water)” indicates the raw voltage calculated by the conductive sensor in the water i.e., where highest moisture levels are recorded. According to an embodiment, this value may be recorded once and used for future calculations and logic processing.

“soilMoistureCond” indicates the soil moisture value for the soil.

At 406, a weighted average of the soil moisture is determined.

According to an embodiment, the weighted average of the soil moisture is determined by the following:

$$soilMoistureAvg = \frac{( soilMoistureCap + soilMoistureCond )}{2}$$

“soilMoistureAvg” indicates the weighted average of the soil moisture based on the soil moisture calculated by the conductive sensor and the soil moisture calculated by the capacitive sensor.

At 408, the total soil moisture is determined based on the conductive soil moisture value and the capacitive soil moisture value.

According to an embodiment, the total soil moisture is determined by the following:

$$\% soilMoistureTot = 100 * ( s o i l M o i stureCap ( 1 - soilMoistureAvg ) + soilMoistureCond ( soilMoistureAvg ) )$$

“% soilMoistureTot” indicates the percentage total soil moisture level in the soil.

According to an embodiment, both the capacitive sensor and the conductive sensor are active in a soil detection operation. The weight accorded to each sensor is adjusted based on the individual sensor measurements and weighing method prestored on the server database.

According to an embodiment, in the calculation operation, a temporary average between the sensors is performed, which is then used to weigh each of the individual values. The values of both soil moisture sensors are then combined using the weighting as shown in Equation 4. In 100% saturated soil, only the conductive sensor is used. In 0% saturated soil, only the capacitive sensor is used. In 50% saturated soil, the two sensors are weighted equivalently.

At 410, the calculated soil moisture is transmitted to the user terminal. The user terminal may be one of the plurality of terminals 116a, 116b, and 116c in FIG. 1.

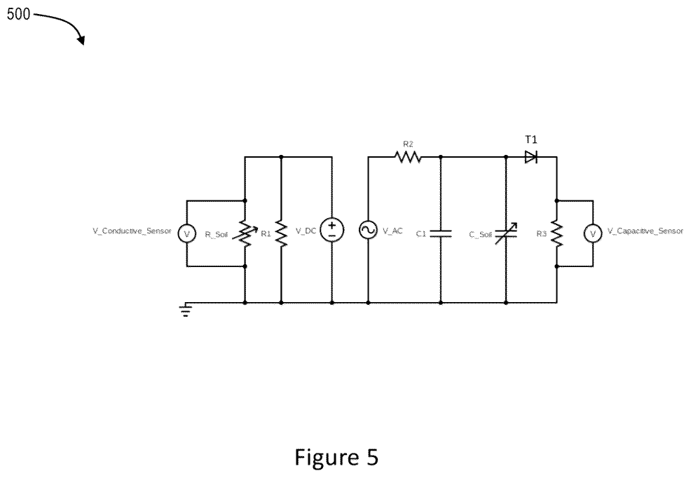

Referring now to FIG. 5, shown therein is a circuit diagram of a device for real-time soil moisture detection and monitoring, according to an embodiment.

The soil moisture detection device includes at least one conductive soil sensor and at least one capacitive soil sensor. By providing a combination of sensors including at least one conductive soil sensor and at least one capacitive oil sensor, enhanced moisture detection capability is achieved for soils ranging from highly saturated to dry or parched.

The conductive soil sensor is configured to measure the moisture content of the soil by performing a DC resistance measurement using a voltage divider. The soil is represented as a variable resistor which has increased resistance as moisture decreases. Performing the DC resistance measurement requires current flow across the two contacts through the soil. While the current is flowing, the oxide is deposited onto the anodic contact on the PCB (Printed Circuit Board). To minimize oxide buildup, the circuit is turned on using a pMOS transistor (p-channel metal-oxide-semiconductor field-effect transistor) for approximately 1 microsecond during each data collection cycle.

According to an embodiment, the soil moisture detection device is connected to a microcontroller. The microcontroller is configured to provide one or more of the data processing, data storage, circuit control, communication, automation, task timing and scheduling functions. For example, an ESP32 microcontroller is connected to the soil moisture detection device.

According to an embodiment, the data collection cycle in the soil moisture detection device is adjusted by the microcontroller. Preferably, the data collection cycle is set at 1 microsecond. The duration of the data collection cycle is configurable and may be adjusted through a software interface or a mobile application communicatively connected to the microcontroller. The circuit activation of 1 microsecond is controlled by the pMOS transistor further connected to the microcontroller. The pMOS transistor on receiving instructions from the microcontroller, is configured to pulled up to logic level high or pulled down to a logic level low (0V) to activate or deactivate the circuit based on the data collection cycle. For example, the 1-microsecond data collection cycle may be e-programmatically changed in the code on the microcontroller.

To minimize oxide buildup, the data collection cycle is activated once every data recording period. For example, the data collection cycle of 1 microsecond is activated once every data recording period of 10 minutes. The data recording period is adjustable through the microcontroller.

“R_Soil” indicates the variable resistance value of the soil.

According to an embodiment, “R1” is 20 k Ohm.

“V_Conductive_Sensor” indicates the voltmeter means to detect the raw voltage by the conductive sensor.

“V_DC” indicates the means to apply the direct current.

According to an embodiment, the capacitive sensor is activated in synchronization with the data collection cycle and data recording period in the conductive sensor.

According to an embodiment, R3 is set as 10 k Ohm, for limiting the current to the analog pin reading the voltage form the circuit. The capacitive soil sensor is configured to measure the moisture content of the soil by performing an AC impedance measurement. This is performed using the change in the circuit impedance caused by soil's variable capacitance (“C_Soil”) to measure the corresponding change in voltage across the resistor R3. The soil is represented as a variable capacitor which has increased impedance as current decreases. “C1” indicates a fixed capacitor.

According to an embodiment, no adjustments are made to R3.

“V_Capacitive_Sensor” indicates the voltmeter means to detect the raw voltage by the capacitive sensor. “T1” indicates a transistor diode.

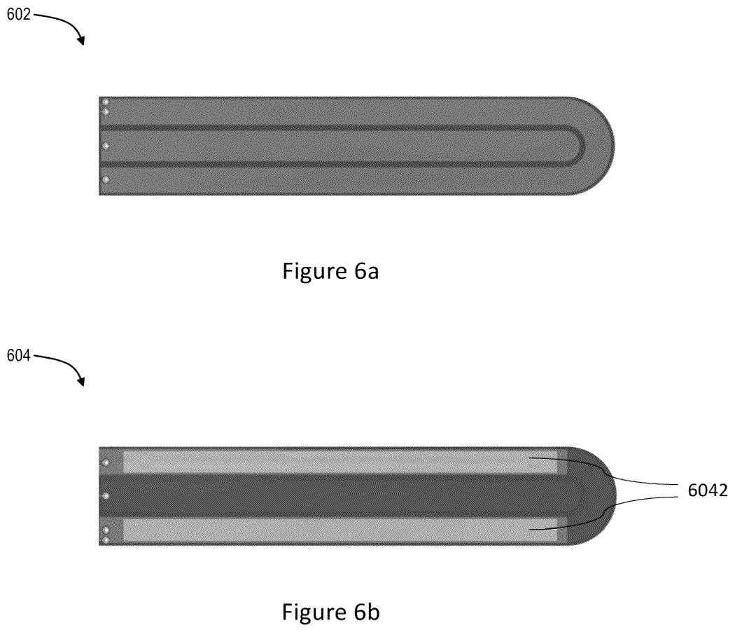

Referring now to FIGS. 6A and 6B, shown therein is a printed circuit board (PCB) of a device for real-time soil moisture detection and monitoring, according to an embodiment.

A schematic diagram of the printed circuit board of the capacitive sensor 602 is provided in FIG. 6A.

The capacitive pads are covered by a solder mask.

According to an embodiment, the variable capacitance of the capacitive sensor increases as a function of soil moisture for larger pad sizes.

A schematic diagram of the printed circuit board of the conductive sensor 602 is provided in FIG. 6B.

The conductive patches are exposed as shown in 6042.

According to an embodiment, the variable resistance of the conductive sensor decreases for larger pad sizes.

The variable resistance of the conductive sensor decreases for larger pad sizes.

While the above description provides examples of one or more apparatus, methods, or systems, it will be appreciated that other apparatus, methods, or systems may be within the scope of the claims as interpreted by one of skill in the art.

1. A soil moisture detection system, the system comprising:
at least one soil moisture detection device including:
a conductive sensor configured to:
detect a conductive soil moisture raw data by a DC resistance measurement on a conductive voltage divider for a soil, wherein the soil operates as a variable resistor wherein a soil resistance is inversely proportional to the soil moisture;
a capacitive sensor configured to
detect a capacitive soil moisture level raw data by an AC impedance measurement on a capacitive voltage divider for the soil, wherein the soil operates as a variable capacitor wherein a soil impedance is inversely proportional to a current;
a network server configured to provide a soil moisture detection; and
a user terminal configured to receive a soil moisture value.

2. The soil moisture detection system of claim 1, wherein the conductive sensor performs DC resistance measurement at a pre-set time interval.

3. The soil moisture detection system of claim 2, wherein the pre-set time interval is provided by a p-channel metal-oxide-semiconductor field-effect transistor.

4. The soil moisture detection system of claim 1, wherein the network server determines a conductive soil moisture level from:
$$soilMoistureCond = \frac{( [ V\_COND @ ( air ) ] - [ V\_COND @ ( raw ) ] )}{( [ V\_COND @ ( air ) - [ V\_COND @ ( water ) ] )}$$
wherein the soilMoistureCond refers to the conductive soil moisture level.

5. The soil moisture detection system of claim 4, wherein the network server determines a capacitive soil moisture level from:
$$soilMoistureCap = \frac{( [ V\_CAP @ ( air ) ] - [ V\_CAP @ ( raw ) ] )}{( [ V\_CAP @ ( air ) - [ V\_CAP @ ( water ) ] )}$$
wherein the soilMoistureCap refers to the capacitive soil moisture level.

6. The soil moisture detection system of claim 5, wherein the network server determines an average soil moisture level from:
$$soilMoistureAvg = \frac{( soilMoistureCap + soilMoistureCond )}{2}$$
wherein the soilMoistureAvg refers to the average soil moisture level.

7. The soil moisture detection system of claim 6, wherein the network server determines a total soil moisture level from:
$$\% soilMoistureTot = 100 * ( s o i l M o i stureCap ( 1 - soilMoistureAvg ) + soilMoistureCond ( soilMoistureAvg ) )$$
wherein the % soilMoistureTot refers to the average soil moisture level.

8. The soil moisture detection system of claim 7, wherein the network server is configured to determine the conductive sensor moisture level in response to a saturated soil.

9. The soil moisture detection system of claim 7, wherein the network server is configured to determine the capacitive sensor moisture level in response to a dry soil.

10. A soil moisture detection device, the device comprising:
a processing unit;
a conductive sensor configured to:
detect a conductive soil moisture raw data by a DC resistance measurement on a conductive voltage divider for a soil, wherein the soil operates as a variable resistor wherein a soil resistance is inversely proportional to the soil moisture; and
a capacitive sensor configured to
detect a capacitive soil moisture level raw data by an AC impedance measurement on a capacitive voltage divider for the soil, wherein the soil operates as a variable capacitor wherein a soil impedance is inversely proportional to a current.

11. The soil moisture detection device of claim 10, wherein the conductive sensor performs DC resistance measurement at a pre-set time interval.

12. The soil moisture detection device of claim 11, wherein the pre-set time interval is provided by a p-channel metal-oxide-semiconductor field-effect transistor.

13. The soil moisture detection system of claim 10, wherein the processing unit determines a conductive soil moisture level from:
$$soilMoistureCond = \frac{( [ V\_COND @ ( air ) ] - [ V\_COND @ ( raw ) ] )}{( [ V\_COND @ ( air ) - [ V\_COND @ ( water ) ] )}$$
wherein the soilMoistureCond refers to the conductive soil moisture level.

14. The soil moisture detection device of claim 13, wherein the processing unit determines a capacitive soil moisture level from:
$$soilMoistureCap = \frac{( [ V\_CAP @ ( air ) ] - [ V\_CAP @ ( raw ) ] )}{( [ V\_CAP @ ( air ) - [ V\_CAP @ ( water ) ] )}$$
wherein the soilMoistureCap refers to the capacitive soil moisture level.

15. The soil moisture detection system of claim 14, wherein the processing unit determines an average soil moisture level from:
$$soilMoistureAvg = \frac{( soilMoistureCap + soilMoistureCond )}{2}$$
wherein the soilMoistureAvg refers to the average soil moisture level.

16. The soil moisture detection system of claim 15, wherein the processing unit determines a total soil moisture level from:
$$\% soilMoistureTot = 100 * ( s o i l M o i stureCap ( 1 - soilMoistureAvg ) + soilMoistureCond ( soilMoistureAvg ) )$$
wherein the % soilMoistureTot refers to the average soil moisture level.

17. The soil moisture detection system of claim 16, wherein the processing unit is configured to:
determine the conductive sensor moisture level in response to a saturated soil; and
determine the capacitive sensor moisture level in response to a dry soil.

18. A soil moisture detection method, the method comprising:
collecting conductive raw voltage data at a conductive sensor by a DC resistance measurement on a conductive voltage divider for a soil, wherein the soil operates as a variable resistor wherein a soil resistance is inversely proportional to the soil moisture;
collecting capacitive raw voltage data at a capacitive sensor by an AC impedance measurement on a capacitive voltage divider for the soil, wherein the soil operates as a variable capacitor wherein a soil impedance is inversely proportional to a current; and
transmitting the conductive raw voltage data and capacitive raw voltage data to a server for determination of a conductive soil moisture level, a capacitive soil moisture level, an average soil moisture level, and a total soil moisture level.

19. The soil moisture detection method of claim 18, the method comprising:
the conductive soil moisture level is determined from:
$$soilMoistureCond = \frac{( [ V\_COND @ ( air ) ] - [ V\_COND @ ( raw ) ] )}{( [ V\_COND @ ( air ) - [ V\_COND @ ( water ) ] )} ;$$
the capacitive soil moisture level is determined from:
$$soilMoistureCap = \frac{( [ V\_CAP @ ( air ) ] - [ V\_CAP @ ( raw ) ] )}{( [ V\_CAP @ ( air ) - [ V\_CAP @ ( water ) ] )} ;$$
the average soil moisture level is determined from:
$$soilMoistureAvg = \frac{( soilMoistureCap + soilMoistureCond )}{2} ;$$
and the total soil moisture level is determined from:
$$\% soilMoistureTot = 100 * ( s o i l M o i stureCap ( 1 - soilMoistureAvg ) + soilMoistureCond ( soilMoistureAvg ) ) ; and$$
wherein the soilMoistureCond refers to the conductive soil moisture level; the soilMoistureCap refers to the capacitive soil moisture level; wherein the soilMoistureAvg refers to the average soil moisture level; and
the % soilMoistureTot refers to the total soil moisture level.

20. The soil moisture detection method of claim 19, wherein the server is configured to:
determine the conductive sensor moisture level in response to a saturated soil; and
determine the capacitive sensor moisture level in response to a dry soil.
